(19) United States
(12) Patent Application Publication
(10) Pub. No.: US 2022/0176016 A1
(43) Pub. Date: Jun. 9, 2022

(54) IMPLANT MATERIAL CONTAINING SURFACE-TREATED AROMATIC POLYETHER KETONE AND MANUFACTURING METHOD THEREFOR

(71) Applicants: NATIONAL UNIVERSITY CORPORATION OKAYAMA UNIVERSITY, Okayama-shi, Okayama (JP); SHINSHU UNIVERSITY, Matsumoto-shi, Nagano (JP); FUKUOKA UNIVERSITY, Fukuoka-shi, Fukuoka (JP)

(72) Inventors: Takumi Okihara, Okayama-shi, Okayama (JP); Yoshiki Takada, Okayama-shi, Okayama (JP); Naoto Saito, Matsumoto City, Nagano (JP); Kaoru Aoki, Matsumoto City, Nagano (JP); Naoyuki Nishimura, Matsumoto City, Nagano (JP); Hisao Haniu, Matsumoto City, Nagano (JP); Shigeaki Moriyama, Fukuoka-shi, Fukuoka (JP); Katsuya Ueda, Matsumoto City, Nagano (JP)

(73) Assignees: NATIONAL UNIVERSITY CORPORATION OKAYAMA UNIVERSITY, Okayama-shi, Okayama (JP); SHINSHU UNIVERSITY, Matsumoto-shi, Nagano (JP); FUKUOKA UNIVERSITY, Fukuoka-shi, Fukuoka (JP)

(21) Appl. No.: 17/599,056

(22) PCT Filed: Mar. 27, 2020
(86) PCT No.: PCT/JP2020/014238

§ 371 Date: Sep. 28, 2021

(30) Foreign Application Priority Data
Mar. 29, 2019 (JP) 2019-066485

(51) Int. Cl. A61L 27/32; A61L 27/18; A61L 27/34; A61L 27/54; C08G 65/48; C08G 65/40; C08J 7/14

(52) U.S. Cl. CPC A61L 27/32; A61L 27/18; A61L 27/34; A61L 27/54; C08J 2371/10; C08G 65/4012; C08J 7/14; A61L 2430/02; A61L 2300/412; C08G 65/48

(57) ABSTRACT

The present invention addresses the problem of providing, by a method in which an expensive manufacturing apparatus is not required, an implant material having osteoconductivity superior to that of an implant material containing an aromatic polyether ketone. The present invention pertains to: said method including immersing an aromatic polyether ketone in a strong base solution in the absence of a calcium ion, and immersing an aromatic polyether ketone, which is obtained by the immersing, in a liquid containing a phosphorus-containing compound; and an implant material obtained by said method.

### TECHNICAL FIELD

The present invention relates to an implant material comprising an aromatic polyetherketone used for vertebral body cages and the like for spine treatment, and a method for producing the same.

### BACKGROUND ART

Implants used for treatment of cervical vertebrae such as vertebral body spacers are generally made of titanium alloy or the like. Titanium alloy has high biocompatibility and high osteoconductivity. However, because it is a metal, it has high rigidity against bones and may destroy bones. For this reason, caution is required regarding the use for elderly people with weak bones. In addition, because it has magnetism, it causes halation during diagnostic imaging such as MRI, and is regarded as one of the interfering factors for accurate diagnostic imaging.

In recent years, vertebral body spacers using PEEK (poly-ether-ether-ketone) that is one of the super engineering plastics have been developed. PEEK is known to be very chemically stable and its rigidity is very close to that of human bones, and the possibility of bone destruction is low as compared with the above-mentioned titanium vertebral body spacers.

On the other hand, it has lower osteoconductivity than titanium, which is a drawback to the titanium alloy vertebral body spacers.

Various efforts have been made so far to improve osteoconductivity.

For example, the following methods have been reported: a method of depositing metal ion plasma on a base material and attaching cells such as osteoblasts to the metal ion plasma (for example, Patent Document 1), a method of mixing with a bioactive fine particle ceramic containing hydroxyapatite (for example, Patent Document 2), and a method of forming a metal oxide adhesive layer on a polymer surface (for example, Patent Document 3) and the like.

In addition, regarding vertebra cages, a vertebra cage has been reported in which an osteoconductive member is filled and integrated in the long axis portion extending through the bone contact surfaces on both sides of a structural member formed of PEEK or the like (for example, Patent Document 4).

Furthermore, there are a report on a method of supplying a source of soluble calcium ions and a source of soluble phosphate ions to a base material at predetermined concentrations (Patent Document 6), and a report on a method for producing a biological implant by immersing a surface foaming body in both of a solution comprising calcium ions and a solution comprising phosphate ions (Patent Document 7), and the like; however, these methods in Patent Documents 6 and 7 are a method of precipitating and coating calcium phosphate, etc. on the surface of a base material, and there is no chemical bond between the base material and phosphorus atoms.

Even when these methods were used, none of them provided the same osteoconductivity as titanium.

In addition, a method of using sodium hydroxide and fluorine gas, when imparting a hydrophilic functional group such as a carboxyl group to the surface of PEEK, has been proposed (Patent Document 5); however, since this production method uses dangerous fluorine gas, a large investment in plant and equipment is required for corrosion resistance and safety of the manufacturing equipment.

Furthermore, although a method of introducing a phosphate group into the surface of PEEK is provided (Non-Patent Document 1), a special technique such as use of plasma is required, and a large investment in plant and equipment is required.

### CITATION LIST

### Patent Documents

[Patent Document 1] JP A No. 2009-542261
[Patent Document 2] JP A No. 2010-246934
[Patent Document 3] JP A No. 2011-500216
[Patent Document 4] JP A No. 2007-512874
[Patent Document 5] JP A No. 2016-209197
[Patent Document 6] WO 2009/095960
[Patent Document 7] JP A No. 4-224747
[Non-Patent Document 1] Colloids and Surfaces B: Biointerfaces, 197, 36-42, 2019. Naoyuki Fukuda, Akira Tsuchiya, Sunarso, Riki Toita, Kanji Tsuru, Yoshihide Mori, Kunio Ishikawa. Surface plasma treatment and phosphorylation enhance the biological performance of poly(ether ether ketone)

### SUMMARY OF THE INVENTION

### Problems to be Solved by the Invention

In order to safely and inexpensively supply an implant material without causing problems of high rigidity, bone destruction, and halation when using titanium, and which has osteoconductivity comparable to that of titanium, the present inventors have come to realize that development of new surface treatment technologies for materials such as PEEK, etc. is necessary. Therefore, an object of the present invention is to provide a practical implant material by surface-treating a material such as PEEK safely and inexpensively.

In an attempt to solve the above-mentioned problems, the present inventors have found that an implant material having sufficient osteoconductivity can be obtained by immersing an aromatic polyetherketone such as PEEK in a strong basic solution such as sodium hydroxide, then immersing the aromatic polyetherketone obtained by said immersion treatment in a solution comprising a phosphorus-containing compound such as a phosphorus oxychloride solution; after conducting further research, the present inventors have completed the present invention.

### Means for Solving the Problems

Therefore, the present invention relates to the following.

[1] A method of producing an implant material comprising a surface-treated aromatic polyetherketone, wherein: the surface treatment comprises immersing an aromatic polyetherketone in a strong base solution in the absence of calcium ions, and immersing the aromatic polyetherketone obtained by said immersion treatment in a solution comprising a phosphorus-containing compound.
[2] The method according to [1], which comprises adsorbing calcium ions on the aromatic polyetherketone surface-treated in the absence of calcium ions.
[3] The method according to [1] or [2], wherein the aromatic polyetherketone is PEEK (poly-ether-ether-ketone) or PEK (poly-ether-ketone).
[4] The method according to any one of [1] to [3], wherein the strong base solution is selected from the group consisting of a lithium hydroxide solution, a sodium hydroxide solution, a potassium hydroxide solution, a rubidium hydroxide solution, a cesium hydroxide solution, a tetraalkylammonium hydroxide solution, a calcium hydroxide solution, a strontium hydroxide solution, a barium hydroxide solution, an europium hydroxide solution, and a thallium hydroxide solution.
[5] The method according to any one of [1] to [4], wherein the strong base solution is a sodium hydroxide solution of 3N or more.
[6] The method according to any one of [1] to [5], wherein the solution comprising a phosphorus-containing compound is selected from the group consisting of phosphorus oxychloride solution, phosphorus trichloride solution, phosphorus oxybromide solution, phosphorus tribromide solution, polyphosphate solution, phosphorus pentachloride solution, pyrophosphate solution, and phosphorous acid diester such as dimethyl chlorophosphite, diethyl chlorophosphite, and propyl chlorophosphite.
[7] The method according to any one of [1] to [6], wherein the surface treatment does not comprise plasma treatment of the aromatic polyetherketone.
[8] An implant material comprising the surface-treated aromatic polyetherketone produced by the method according to any one of [1] to [7].
[9] The implant material according to [8], wherein the surface-treated aromatic polyetherketone has phosphorus atoms of 0.11 wt % or more in the surface composition.
[10] An implant material which comprises an aromatic polyetherketone having phosphorus atoms of 0.11 wt % or more in the surface composition and comprising a phosphate group formed on the surface.
[11] The implant material according to [10], wherein calcium ions are adsorbed on the surface on which the phosphate group is formed.
[12] The implant material according to [10] or [11], wherein the surface of the aromatic polyetherketone on which the phosphate group is formed is not plasma-treated.

### Advantageous Effects of the Invention

The present invention can provide an implant material having better osteoconductivity than surface-untreated aromatic polyetherketone and also having osteoconductivity comparable to that of titanium. Furthermore, during the follow-up observation using MRI after treatment, titanium implants have a problem of halation, which makes the observation difficult; however, the implant material of the present invention does not cause such a problem, and follow-up observation after treatment can be made together with accurate diagnostic imaging. Moreover, the present invention can produce implant materials by a simple method using inexpensive materials without using expensive manufacturing apparatus or manufacturing equipment such as equipment for using fluorine gas, and therefore, it is possible to provide implant materials for a wide range of applications including implant materials for the treatment cervical vertebrae such as vertebral body spacers and implant materials for dental treatment.

Furthermore, since the method of the present invention can produce an implant material without going through a step of immersing in calcium ions, the implant material can be provided extremely easily. Moreover, the method of the present invention is excellent in terms of the following viewpoints: that is, since the method of the present invention can produce an implant material by a method that does not comprise plasma treatment, equipment for plasma treatment is not required and the number of steps is reduced, and the implant material can be produced inexpensively and easily. Furthermore, the method comprising plasma treatment causes large surface damage, whereas the method of the present invention uses a base such as sodium hydroxide, and the surface damage is minimal as compared with plasma treatment. The implant material obtained by the production method of the present invention exhibits high antibacterial activity and can be used with a lower risk of infection. In addition, the implant material obtained by the production method of the present invention exhibits a higher bone contact ratio and a higher maximum stress during punching from the bone when actually used in vivo, so that the implant material can withstand more intense exercise.

### BRIEF DESCRIPTION OF DRAWINGS

FIG. 1 is a graph showing comparison of relative proliferation rates of osteoblast-like cells, when the untreated PEEK material is used and when the implant material obtained by the production method of the present invention is used.

FIG. 2 shows rabbit femoral implant implantation test of Product 1 of the present invention and PEEK.

FIG. 3 shows the results of histopathological examination at 4 weeks of implantation of Product 1 of the present invention and PEEK.

FIG. 4 shows the results of histopathological examination at 4 weeks of implantation of Product 1 of the present invention and PEEK.

FIG. 5 shows the results of histopathological examination at 4 weeks of implantation of Product 1 of the present invention and PEEK.

FIG. 6 shows the results of histopathological examination at 4 weeks of implantation of Product 1 of the present invention and PEEK.

FIG. 7 shows bone contact ratios of Product 1 of the present invention and PEEK obtained from the rabbit femoral implant implantation test.

FIG. 8 shows the results of punching test by a mechanics testing machine of Product 1 of the present invention and PEEK at 4 weeks of implantation.

FIG. 9 shows the results of punching test by a mechanics testing machine of Product 1 of the present invention and PEEK at 4, 8 and 12 weeks of implantation.

### EMBODIMENTS FOR CARRYING OUT THE INVENTION

The method of producing an implant material comprising a surface-treated aromatic polyetherketone of the present invention comprises immersing an aromatic polyetherketone in a strong base solution, and immersing the aromatic polyetherketone obtained by said immersion treatment in a solution comprising a phosphorus-containing compound.

The aromatic polyetherketone that can be used in the present invention is not particularly limited as long as it has a linear polymer structure in which benzene ring is bonded by ether and ketone. For example, PEEK, PEK (poly-ether-ketone) and the like can be used; from the viewpoint of medical usage record, use of PEEK is preferable.

In the present invention, the form of the aromatic polyetherketone is not particularly limited as long as it can be used as an implant material. For example, it may be machined by a combined lathe, a machining center or the like.

In the production method of the present invention, the surface treatment may be performed in the absence of calcium ions.

In addition, in the production method of the present invention, the surface treatment may not comprise plasma treatment of the aromatic polyetherketone.

The strong base solution used in the present invention is not particularly limited as long as it is a solution having a basicity capable of introducing a functional group that can be substituted with a substituent having a phosphorus atom such as a phosphate group into the immersed aromatic polyetherketone. Examples of such a functional group include a hydroxyl group, an alkoxy group, and a siloxy group. In addition, in one embodiment, the strong base solution used in the present invention does not contain calcium ions.

Specific examples of strong base solution include a sodium hydroxide aqueous solution, a lithium hydroxide aqueous solution, a potassium hydroxide aqueous solution, a rubidium hydroxide aqueous solution, a cesium hydroxide aqueous solution, a tetraalkylammonium hydroxide aqueous solution, a calcium hydroxide aqueous solution, a strontium hydroxide aqueous solution, a barium hydroxide aqueous solution, an europium hydroxide aqueous solution, and a tallium hydroxide aqueous solution, etc. By using these aqueous solutions, a hydroxyl group can be introduced into the aromatic polyether. The concentration of said strong base solution is not particularly limited as long as a functional group can be introduced, and is preferably 3N or more, more preferably 5N or more. Said strong base solution is preferably a sodium hydroxide solution having a concentration of 3N or more, more preferably 5N or more.

Furthermore, as a strong base solution, the following solutions may be used: a solution of sodium alkoxide such as sodium methoxide, sodium ethoxide, and sodium butoxide, a solution of lithium alkoxide such as lithium methoxide, lithium ethoxide, and lithium butoxide, a solution of potassium alkoxide such as potassium methoxide, potassium ethoxide, and potassium butoxide, and a solution of sodium alkylsilanolate such as sodium trimethylsilanolate, sodium triethylsilanolate, and sodium tripropylsilanolate, a solution of lithium alkylsilanolate such as lithium trimethylsilanolate, lithium triethylsilanolate, and lithium tripropylsilanolate. Examples of solvent that can be used in said solution of alkoxide or alkylsilanolate include alcohols such as methanol, ethanol, propanol and butanol, THF, dichloromethane, chloroform, DMF, DMSO, acetonitrile, water and any combinations thereof. When these strong base solutions are used, an alkoxy group or a siloxy group may be introduced into the aromatic polyether.

The functional group introduced as described above, such as a hydroxyl group, an alkoxy group and a siloxy group, can be used as a functional group that can be substituted with a substituent having a phosphorus atom.

For example, after a hydroxyl group is introduced, by directly applying a solution comprising a phosphorus-containing compound, it can be substituted with a substituent having a phosphorus atom, such as a phosphate group. In addition, an alkoxy group and a siloxy group can be, by applying a solution comprising a phosphorus-containing compound after conversion to a hydroxyl group, substituted with a substituent having a phosphorus atom such as a phosphate group. An alkoxy group and a siloxy group can be converted to a hydroxyl group by treatment with, for example, a sodium hydroxide aqueous solution, a lithium hydroxide aqueous solution, a potassium hydroxide aqueous solution, a rubidium hydroxide aqueous solution, a cesium hydroxide aqueous solution, a tetraalkylammonium hydroxide aqueous solution, a calcium hydroxide aqueous solution, a strontium hydroxide aqueous solution, a barium hydroxide aqueous solution, an europium hydroxide aqueous solution, and a tallium hydroxide aqueous solution; an aqueous solution of hydrochloric acid, sulfuric acid and nitric acid; an aqueous solution, an alcoholic solution, and a THF solution of fluoride reagents such as tetrabutylammonium fluoride, sodium fluoride, potassium fluoride, rubidium fluoride and cesium fluoride, etc.

The strong base solution may be used alone or in combination of two or more, provided that a functional group that can be substituted with a substituent having a phosphorus atom such as a phosphate group can be introduced into the aromatic polyether ether ketone. Preferably, a sodium hydroxide aqueous solution, a lithium hydroxide aqueous solution, and a potassium hydroxide aqueous solution are used, and more preferably a sodium hydroxide solution is used alone.

The solution comprising a phosphorus-containing compound used in the method of producing an implant material of the present invention is not particularly limited as long as phosphorus atoms can be introduced into the aromatic polyetherketone. In addition, in one embodiment, the solution comprising a phosphorus-containing compound used in the present invention does not contain calcium ions.

The solution comprising a phosphorus-containing compound used in the present invention may contain, for example, a phosphorus-containing compound selected from the group consisting of phosphorus oxychloride solution, phosphorus trichloride solution, phosphorus oxybromide solution, phosphorus tribromide solution, polyphosphate solution, phosphorus pentachloride solution, pyrophosphate solution, and phosphorous acid diester chlorides such as dimethyl chlorophosphite, diethyl chlorophosphite, and propyl chlorophosphite. In addition, the solution comprising a phosphorus-containing compound of the present invention may be a stock solution of a phosphorus-containing compound when the phosphorus-containing compound is a liquid under the conditions of immersion treatment.

Substituents having a phosphorus atom introduced into aromatic polyetherketone may be, for example, phosphoric acid, phosphorous acid, phosphonic acid, phosphonous acid, phosphoric acid ester, phosphorous acid ester, phosphonic acid ester, phosphonous acid ester, etc. The introduced phosphorous acid, phosphonic acid, phosphonous acid, phosphoric acid ester, phosphorous acid ester, phosphonic acid ester, phosphonous acid ester, etc. may be optionally converted to phosphoric acid in air or in an aqueous solution.

The method of producing an implant material of the present invention may comprise, after the immersion treatment in the phosphorus oxychloride solution, a post-treatment for removing impurities derived from a solution comprising a phosphorus-containing compound such as phosphorus oxychloride adhering to the implant material. In addition, the method of producing an implant material of the present invention may comprise making calcium ions to adsorb at the same time as the post-treatment for removing impurities derived from a supply agent, or after said post-treatment.

The solution used for the post-treatment is not particularly limited as long as it can remove impurities. For example, a sodium hydroxide solution, a lithium hydroxide solution, a potassium hydroxide solution, and a calcium hydroxide solution are used. As the solution used for the post-treatment, a calcium ion-containing solution may be selected from the viewpoint of making calcium ions to adsorb at the same time as the post-treatment.

The temperature in the post-treatment is not particularly limited as long as impurities can be removed. It is carried out at 20° C. to 80° C., preferably 40° C. to 80° C., and more preferably 60° C. to 80° C.

The post-treatment time is not particularly limited. For example, it is carried out for 1 hour to 24 hours, preferably 6 hours to 24 hours, and more preferably 12 hours to 24 hours.

The method of producing an implant material of the present invention may comprise a washing step. The solution used for such washing is not particularly limited as long as it can be used for medical purposes. For example, it is carried out with pure water.

The temperature in the washing is not particularly limited as long as the implant material is sufficiently washed. Washing is carried out at 20° C. to 80° C., preferably 40° C. to 80° C., and more preferably 60° C. to 80° C. Washing may be performed multiple times.

The temperature in the immersion treatment with a strong base solution of the present invention is not particularly limited as long as a functional group such as a hydroxyl group can be introduced. For example, it is carried out at 20° C. to 80° C., preferably 40° C. to 80° C., and more preferably 40° C. to 60° C.

The temperature in the immersion treatment with a solution comprising a phosphorus-containing compound of the present invention is not particularly limited as long as a substituent having a phosphorus atom can be introduced. For example, it is carried out at 0° C. to 40° C., preferably 10° C. to 30° C., and more preferably 10° C. to 20° C.

In the method of producing an implant material of the present invention, the immersion treatment time with a strong base solution is not particularly limited as long as a functional group such as a hydroxyl group can be introduced. For example, it is carried out for 1 hour to 24 hours, preferably 6 hours to 24 hours, and more preferably 12 hours to 24 hours.

In the method of producing an implant material of the present invention, the immersion treatment time in a solution comprising a phosphorus-containing compound is not particularly limited as long as a substituent having a phosphorus atom can be introduced. For example, it is carried out for 1 hour to 6 hours, preferably 1 hour to 4 hours, and more preferably 1 hour to 2 hours.

In the method of producing an implant material of the present invention, optionally, prior to the immersion treatment with a solution comprising a phosphorus-containing compound, the water present on the surface of the aromatic polyether ether ketone after the immersion treatment with a strong base solution may be removed. The method for removing the water is not particularly limited, and examples thereof include wiping, air drying, vacuum drying, heat drying, and any combinations thereof.

The implant material obtained by the method of producing an implant material of the present invention has phosphorus atoms of preferably 0.11 wt % or more, and more preferably 0.41 wt % or more in terms of surface composition.

The phosphorus atom contained in the material obtained by the production method of the present invention may be derived from phosphoric acid or phosphate.

The material obtained by the production method of the present invention may have an atom other than the phosphorus atom, for example, a chlorine atom and a sodium atom.

The material obtained by the production method of the present invention may have an alkali metal adsorbed on the surface, and such alkali metal is not particularly limited as long as it can be used for medical purposes. For example, of lithium (Li), sodium (Na), potassium (K), rubidium (Rb), cesium (Cs), and francium (Fr), it is preferable that sodium or calcium is adsorbed, and more preferably calcium is adsorbed.

Adsorption of these alkali metals can be performed before the surface treatment, at the same time as the surface treatment, or after the surface treatment. For example, after the surface treatment, an alkali metal can be adsorbed on the surface on which phosphate groups are formed. Therefore, in one embodiment of the implant material of the present invention, calcium ions are adsorbed on the surface on which phosphate groups are formed.

Furthermore, adsorption of an alkali metal can be performed, for example, by immersing the base material in a solution comprising the alkali metal ions to be adsorbed. For example, when sodium is adsorbed as an alkali metal, it can be carried out by immersing the base material in a solution such as a sodium hydroxide aqueous solution having a predetermined concentration for a certain period of time. In addition, for example, when calcium is adsorbed as an alkali metal, it can be carried out by immersing the base material in a solution such as a calcium hydroxide aqueous solution of a predetermined concentration for a certain period of time.

In the implant material of the present invention, the surface of the aromatic polyetherketone on which phosphate groups are formed does not have to be plasma-treated.

The implant material of the present invention can be used for cervical vertebrae or for dentistry. In addition, before being applied into the body, osteoblasts may be adhered and used.

### EXAMPLES

### 1. Raw Materials

### PEEK Material

As the PEEK material used, an implant grade i2P manufactured by Daicel-Evonik Ltd. was used. This material was machined into a test piece having a diameter of 14 mm and a thickness of 2 mm.

Surface treatment material Sodium hydroxide: special grade reagent manufactured by Nacalai Tesque, Inc.

Phosphorus oxychloride: manufactured by Nacalai Tesque, Inc.

### 2. Each Test Example

### Test Example 1

Step 1: Preparation of sodium hydroxide aqueous solution: The sodium hydroxide aqueous solution was adjusted so as to be 5N. The above test piece was immersed in this solution for 24 hours.

Step 2: The test piece obtained in Step 1 was taken out, the water on the surface was wiped off, and the test piece was immersed in a phosphorus oxychloride solution (stock solution) for 24 hours.

Step 3: Adjustment of sodium hydroxide aqueous solution: The sodium hydroxide aqueous solution was adjusted so as to be 1N. The above test piece was immersed in this solution for 24 hours. After that, it was washed with pure water.

All of the above steps 1 to 3 were performed at room temperature (20° C.)

The PEEK material whose surface was phosphorylated by the treatments of the above steps 1 to 3 was obtained (Product 1).

### Test Example 2

Adjustment of a sodium hydroxide aqueous solution in step 1 was performed in the same manner as in Test Example 1, except that the sodium hydroxide aqueous solution was adjusted to 3N, to obtain a PEEK material whose surface was phosphorylated (Product 2).

### Test Example 3

Adjustment of a sodium hydroxide aqueous solution in step 1 was performed in the same manner as in Test Example 1, except that the sodium hydroxide aqueous solution was adjusted to 1N, to obtain a PEEK material whose surface was phosphorylated (Product 3).

### 3. Engineering and Biological Evaluation

### 3-1 Engineering Evaluation

The following analysis was carried out to confirm the presence of phosphorus atoms on the surface.

The presence of phosphate groups on the surface was confirmed using an electron microscope (VE-9800 EDX manufactured by KEYENCE CORPRATION: EDAX-Genesis manufactured by AMETEK, Inc.). Analytical conditions are as follows: Acceleration voltage 8 kV/Magnification 100×.

The results are shown in Table 1.

TABLE 1

| | Quantitative study of phosphate group | | | |
|---|---|---|---|---|
| Sample | P/wt % | C/wt % | 0/wt % | Na/wt % |
| Product 1 | 0.41 | 84.69 | 14.06 | 0.17 |
| (5N) | | | | |
| Product 2 | 0.11 | 86.43 | 13.26 | 0.04 |
| (3N) | | | | |
| Product 3 | 0.03 | 86.66 | 13.13 | 0.01 |
| (1N) | | | | |

In Product 1 and Product 2, sufficient amounts of phosphorus component could be confirmed. In contrast, in Product 3, although it was a small amount of 0.03 wt %, the presence of phosphorus component could be confirmed. In all the products, phosphorus atoms could be confirmed to be introduced into PEEK, and introduction of phosphate groups was presumed. The reason why the total of each atom is not 100% in Table 1 is considered to be the presence of impurities, etc.

### 3-2. Biological Evaluation

### 3-2-1. Relative Evaluation of Cell Proliferation Rate

Relative evaluation of cell proliferation rate was performed using the evaluation method shown below (MC3T3-E1 cell proliferation promotion test).

Cell: MC3T3-E1 (model number: RCB1126, Lot: 64, Riken BioResource Research Center)

Medium: α-minimum essential medium (model number: 21444-05, Lot: L8H5662, Nacalai Tesque, Inc.) was prepared to contain 10% fetal bovine serum (model number: S1820-500, Lot: S1820-500, Biowest), to which Penicillin-Streptomycin-Amphotericin B Suspension (×100) (model number: 161-23181, Lot: APG7006, Wako) was added in an amount of 1/100.

Reagent: Dulbecco's phosphate buffered saline (−) (model number: 045-29795, Lot: ECR7015, Wako), alamarBlue® cell viability reagent (model number: BUF012B, Lot: 146809, Bio-Rad Laboratories, Inc.)

### <Evaluation Procedure>

1) Each of γ-sterilized products was brought into close contact with the bottom of a well of a 24-well culture plate. In addition, a well that does not contain the product was set as the control group.
2) A cell suspension of MC3T3-E1 cells prepared in a medium so as to have a concentration of 4.0×10⁴ cells/well was added into the well, and the cells were cultured at 37° C. in a 5% CO₂ incubator for 1 day.
3) After culturing for 1 day, the medium was aspirated, the alamarBlue® cell viability reagent diluted to 10% using Dulbecco's phosphate buffered saline (−) was added, and the mixture was added to each well and reacted for 1 hour. In addition, a reagent blank was set in which only the reaction solution was added to the well without seeding of cells.
4) After 1 hour, 100 μl of the reaction solution in each well was transferred to a 96-well assay plate, and the fluorescence intensity was measured at 535/590 nm of Ex/Em using Plate Reader AF2200 (Serial No. 1307005705, Eppendorf AG). Cell proliferation was calculated by: cell viability (%)=100×(sample value-reagent blank value)/(control group value−reagent blank value).
5) The reaction solution in 24 wells was aspirated, the medium was replaced with a fresh medium, and it was cultured at 37° C. in a 5% CO₂ incubator for another 3 days, and evaluated in the same procedure as in 3) to 4) above.

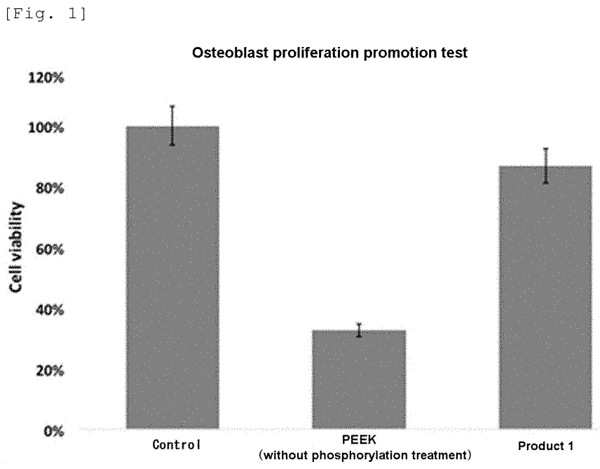

FIG. 1 shows a comparison of relative proliferation rates.

From the figure, it can be seen that the proliferation rate of MC3T3-E1 is significantly increased compared to that of the untreated one.

Therefore, it was found that the product of the present invention had a sufficient osteoblast proliferation promoting effect as an implant material and had sufficient osteoblast conductivity.

### 3-2-2. Antibacterial Test

The antibacterial activity values against S. aureus NBRC of Product 1 of the present invention and PEEK were measured according to JIS 22801, as follows.

After placing the product of the present invention into a sterile petri dish, a test bacterial solution containing the strain of S. aureus NBRC was dropped, and a lid was put on the petri dish. The petri dish was then cultured for 24 hours. Subsequently, the product of the present invention was washed out to an agar medium using a SCDLP medium, and the SCDLP medium was incubated on the agar medium for 48 hours, then the bacteria were collected and viable cell count was measured.

After placing PEEK in a sterile petri dish, a test bacterial solution containing the strain of S. aureus NBRC was dropped and a lid was put on the petri dish. The petri dish was then cultured for 24 hours. Subsequently, the product of the present invention was washed out to an agar medium using a SCDLP medium, and the SCDLP medium was incubated on the agar medium for 48 hours, then the bacteria were collected and the viable cell count was measured.

As a control, after placing the unprocessed product in a sterile petri dish, a test bacterial solution containing the strain of S. aureus NBRC was dropped and a lid was put on the petri dish. The petri dish was then cultured for 24 hours. Subsequently, the product of the present invention was washed out to an agar medium using a SCDLP medium, and the SCDLP medium was incubated on the agar medium for 48 hours, then the bacteria were collected and the viable cell count was measured.

Similarly, the antibacterial activity values against E. coli NBRC 3972 of Product 1 of the present invention and PEEK were measured as follows.

The antibacterial activity values against S. aureus NBRC of the product of the present invention and PEEK were measured as follows.

After placing the product of the present invention in a sterilized petri dish, a test bacterial solution containing the strain of E. coli NBRC 3972 was dropped and a lid was put on the petri dish. The petri dish was then cultured for 24 hours. Subsequently, the product of the present invention was washed out to an agar medium using a SCDLP medium, and the SCDLP medium was incubated on the agar medium for 48 hours, then the bacteria were collected and the viable cell count was measured.

After placing PEEK in a sterile petri dish, a test bacterial solution containing the strain of E. coli NBRC 3972 was dropped and a lid was put on the petri dish. The petri dish was then cultured for 24 hours. Subsequently, the product of the present invention was washed out to an agar medium using a SCDLP medium, and the SCDLP medium was incubated on the agar medium for 48 hours, then the bacteria were collected and the viable cell count was measured.

As a control, after placing the unprocessed product in a sterile petri dish, a test bacterial solution containing the strain of E. coli NBRC 3972 was dropped and a lid was put on the petri dish. The petri dish was then cultured for hours. Subsequently, the product of the present invention was washed out to an agar medium using a SCDLP medium, and the SCDLP medium was incubated on the agar medium for 48 hours, then the bacteria were collected and the viable cell count was measured. The antibacterial activity values against E. coli NBRC 3972 and S. aureus NBRC of the product of the present invention and PEEK were determined from the following formula; the results are as shown in Table 2.

TABLE 2

| | Product 1 | PEEK |
|---|---|---|
| S. aureus | 1.9 | 0 |
| E. coli | 1.2 | −1.4 |

From the values in Table 2, it was clarified that the product of the present invention exhibited higher antibacterial activity values than PEEK for both E. coli NBRC 3972 and S. aureus NBRC.

### 3-2-3. Rabbit Femoral Implant Implantation Test

Using Product 1 of the present invention, a rabbit femoral implant implantation test was performed. The thighs and lower legs of Japanese white rabbits (Japan SLC Inc., male, weighing 2.5 to 3.5 kg) subjected to isoflurane inhalation anesthesia were shaved with a hair clipper. A longitudinal incision was made in the skin from the knee to the lateral side of the thigh with a scalpel, and the intermuscular septum on the lateral side of the thigh was peeled off to reach the lateral part of the femur. Then, a bone hole was made from the outside of the femoral condyle with a drill having a diameter of 5 mm. The inside of the bone hole was washed with physiological saline, and a groove was made in the product of the present invention to make a columnar implant having a diameter of 5 mm and a height of 15 mm, which was inserted in the bone hole. The operation was completed by suturing the deep fascia of the thigh and the skin.

Next, at 4, 8 and 12 weeks after the implantation, the rabbits were euthanized by auricular vein injection of 1% lidocaine. The skin, muscles, and ligaments of the thigh and lower leg were peeled off to expose the bone, and the proximal diaphysis of the femur was cut with a saw to collect the femur. The collected femur was fixed by immersion in a 10% formalin solution.

In the formalin-fixed rabbit femur, only the condyle containing the implant was excised from the diaphysis with a saw. The collected femoral condyle was resin-embedded using an osteoresin embedding kit (FUJIFILM Wako Pure Chemical Corporation). The resin-embedded femoral condyle was sliced with Saw Microtome for polished specimens (5P1600, Leica Microsystems). The specimens were stained with Mayer's hematoxylin (FUJIFILM Wako Pure Chemical Corporation) and Eosin (70% alcohol-soluble) (FUJIFILM Wako Pure Chemical Corporation) and evaluated histopathologically with an optical microscope.

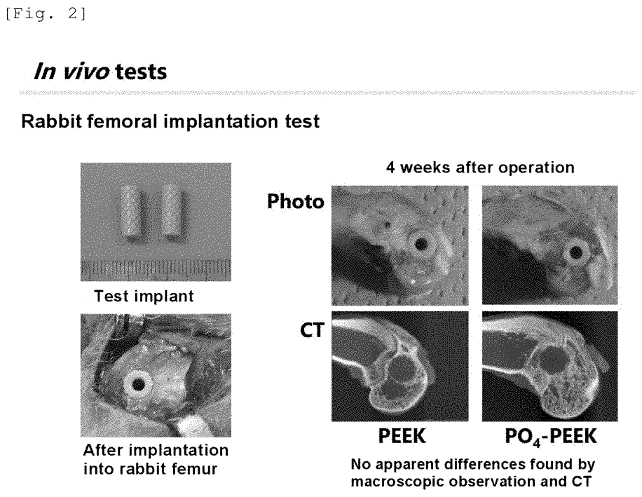

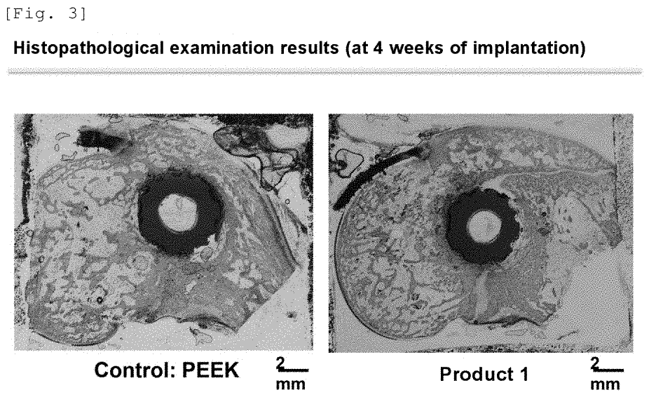

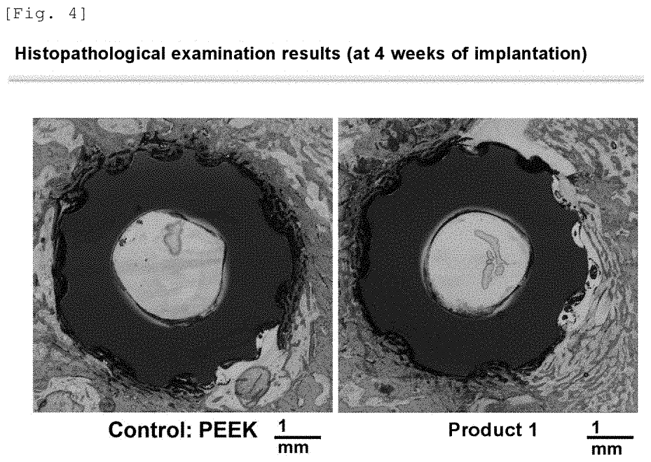

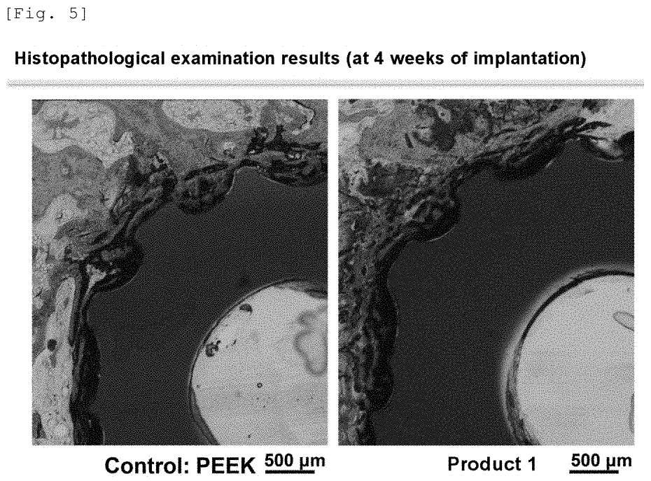

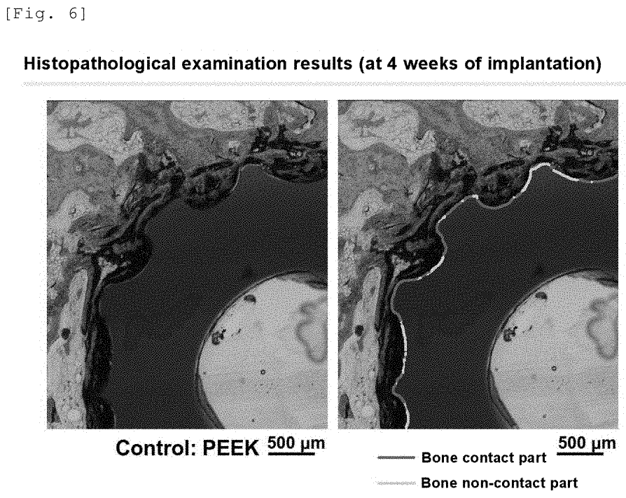

FIG. 2 shows implants for the test (the product of the present invention and untreated PEEK) used in the rabbit femoral implant implantation test, a photograph of the femoral implant implantation, and photographs and CT images weeks after implantation. The results of the histopathological examination are shown in FIGS. 3 to 6.

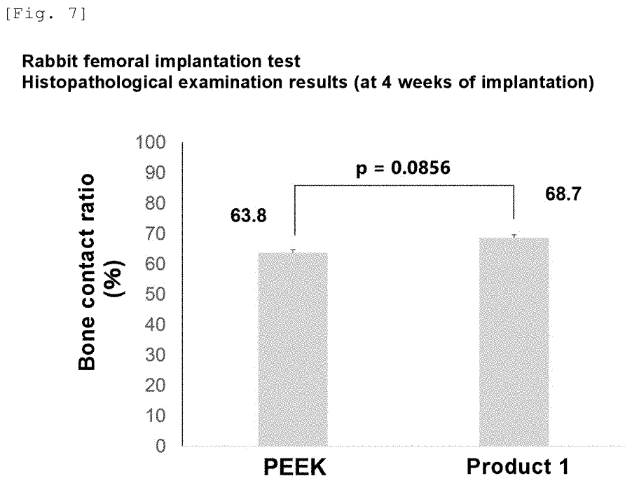

The bone contact ratios of the product of the present invention and PEEK 4 weeks after implantation were measured and found to be 68.7% and 63.8%, respectively, and the product of the present invention showed a higher bone contact ratio than PEEK (FIG. 7).

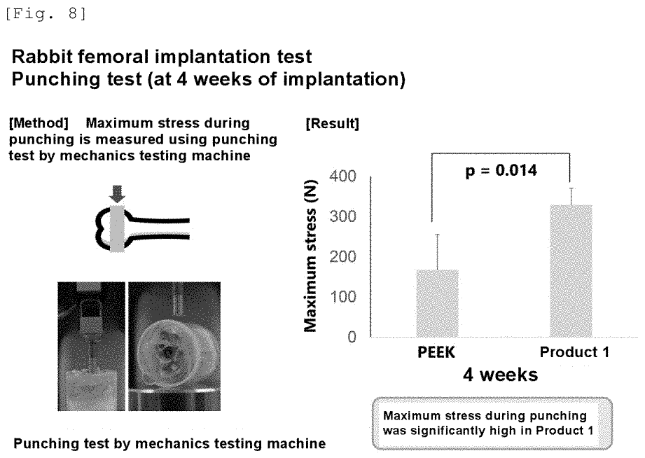

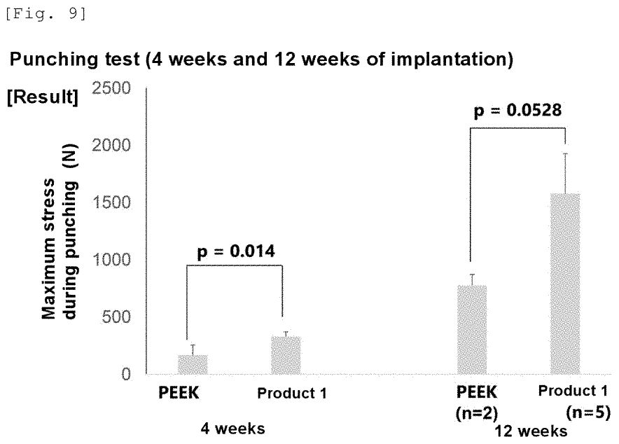

Furthermore, at 4, 8 and 12 weeks after implantation, as shown in FIG. 8, Product 1 of the present invention and the untreated PEEK were punched out by a mechanics testing machine and the maximum stress during punching was respectively measured. The results are shown in FIG. 9.

It was clarified that the maximum stresses during punching of both the product of the present invention and PEEK were stronger at 12 weeks than those at 4 weeks; and at 12 weeks, the maximum stress during punching of the product of the present invention was stronger than that of PEEK. In addition, since the maximum stress during punching increases with time, it can be predicted from the histopathological structure that the bond will be stronger by long-term implantation such as 8 weeks and 12 weeks.

Regarding said implant material, while the introduction of phosphorus atoms into PEEK has been confirmed, there are circumstances in which it is impossible or difficult to directly identify phosphorus atoms due to the structure or characteristics of the object. That is, it may be predicted that the introduced phosphorus atom exists as a phosphate group in the structure of the PEEK, and that it is introduced at the ortho-position or meta-position of the ether oxygen atom of the PEEK, or both; however, in order to confirm such prediction, it is necessary to analyze at least the outermost surface structure of the polymer by XPS (photoelectron spectroscopy), Auger spectroscopy or the like, and to identify the molecular structure thereof. In addition, it is impossible to achieve complete identification by these spectroscopic methods alone. Furthermore, even if the above analysis can be achieved, further analysis is required for the correlation between the outermost surface structure obtained by such analysis and the biological properties; and therefore, it should be said that direct identification of the structure or properties of the implant material of the present invention is extremely difficult or impossible.

1. A method of producing an implant material comprising a surface-treated aromatic polyetherketone, wherein:
the surface treatment of said surface-treated aromatic polyetherketone comprises immersing an aromatic polyetherketone in a strong base solution of 3N or more in the absence of calcium ions, and immersing the resulting aromatic polyetherketone in a solution containing a phosphorus-containing compound.

2. The method according to claim 1, which comprises adsorbing calcium ions on the aromatic polyetherketone surface-treated in the absence of calcium ions.

3. The method according to claim 1, wherein the aromatic polyetherketone is PEEK (poly-ether-ether-ketone) or PEK (poly-ether-ketone).

4. The method according to claim 1, wherein the strong base solution is selected from the group consisting of a lithium hydroxide solution, a sodium hydroxide solution, a potassium hydroxide solution, a rubidium hydroxide solution, a cesium hydroxide solution, a tetraalkylammonium hydroxide solution, a calcium hydroxide solution, a strontium hydroxide solution, a barium hydroxide solution, an europium hydroxide solution, and a thallium hydroxide solution.

5. The method according to claim 1, wherein the strong base solution is a sodium hydroxide solution of 3N or more.

6. The method according to claim 1, wherein the solution containing a phosphorus-containing compound is selected from the group consisting of phosphorus oxychloride solution, phosphorus trichloride solution, phosphorus oxybromide solution, phosphorus tribromide solution, polyphosphate solution, phosphorus pentachloride solution, pyrophosphate solution, and phosphorous acid diester chlorides such as dimethyl chlorophosphite, diethyl chlorophosphite, and propyl chlorophosphite.

7. The method according to claim 1, wherein the surface treatment does not comprise any plasma treatment of the aromatic polyetherketone.

8. The method according to claim 1, wherein the strong base solution is a sodium hydroxide solution of 5N or more and the solution containing a phosphorus-containing compound is a phosphorus oxychloride solution.

9. An implant material comprising the surface-treated aromatic polyetherketone produced by the method according to claim 1.

10. The implant material according to claim 9, wherein the surface-treated aromatic polyetherketone has phosphorus atoms of 0.11 wt % or more in the surface composition.

11. The implant material according to claim 9, wherein the implant material comprises the aromatic polyetherketone comprising a phosphate group formed on the surface.

12. The implant material according to claim 11, wherein calcium ions are adsorbed on the surface on which the phosphate group is formed.

13. The implant material according to claim 11, wherein the surface of the aromatic polyetherketone on which the phosphate group is formed is not plasma-treated.
